(19) United States
(12) Patent Application Publication
(10) Pub. No.: US 2012/0323058 A1
(43) Pub. Date: Dec. 20, 2012

(54) PROCESS FOR USING LAYERED SPHERE CATALYST

(75) Inventors: Gregory J. Gajda, Mt. Prospect, IL (US); Antoine Negiz, Wilmette, IL (US); John Joseph Senetar, Naperville, IL (US); Mark G. Riley, Hinsdale, IL (US); Erik Holmgreen, Racine, WI (US); Bryan K. Glover, Algonquin, IL (US)

(73) Assignee: UOP, LLC, Des Plaines, IL (US)

(21) Appl. No.: 13/161,309

(22) Filed: Jun. 15, 2011

(51) Int. Cl. C07C 5/09

(52) U.S. Cl. 585/260

(57) ABSTRACT

One exemplary embodiment can be a process for selective hydrogenation of acetylenes and diolefins to olefins. The process can include contacting a feedstream having olefins, acetylenes and diolefins with a layered catalyst at reaction conditions. Thus, the process may include creating an output stream with a reduced amount of acetylenes and diolefins. Generally, the layered catalyst has an inner core including an inert material, an outer layer, including a metal oxide, bonded to the inner core, and a metal, which is an International Union of Pure and Applied Chemistry Group 8-10 metal, deposited on the outer layer. Usually, the layered catalyst has an accessibility index of about 3—about 500, a void space index about 0—about 1, or both an accessibility index of about 3—about 500 and a void space index of about 0—about 1.

### FIELD OF THE INVENTION

This invention generally relates to a process for using a layered catalyst.

### BACKGROUND OF THE INVENTION

Platinum based catalysts are used for numerous hydrocarbon conversion processes. In many applications promoters and modifiers are also used. One such hydrocarbon conversion process is the dehydrogenation of hydrocarbons, particularly alkanes such as isobutane, which are converted to isobutylene. Suitable catalyst may include a platinum metal, a tin oxide component and a germanium oxide component with all components uniformly dispersed throughout the alumina support. Alternatively, a catalyst having a base support deposited thereon a layer of a catalytic metal oxide or a combination of a catalytic metal oxide and an oxide support can also be utilized. Further still, a catalyst having a catalytically inert core material deposited and bonded to a thin shell of material containing active sites may be used. However, it is desirable to provide a catalyst and process with improved activity and selectivity for hydrogenating acetylene compounds.

### SUMMARY OF THE INVENTION

One exemplary embodiment can be a process for selective hydrogenation of acetylenes and diolefins to olefins. The process can include contacting a feedstream having olefins, acetylenes and diolefins with a layered catalyst at reaction conditions. Thus, the process may include creating an output stream with a reduced amount of acetylenes and diolefins. Generally, the layered catalyst has an inner core including an inert material, an outer layer, including a metal oxide, bonded to the inner core, and a metal, which is an International Union of Pure and Applied Chemistry Group 8-10 metal, deposited on the outer layer. Usually, the layered catalyst has an accessibility index of about 3—about 500, a void space index about 0—about 1, or both an accessibility index of about 3—about 500 and a void space index of about 0—about 1.

Another exemplary embodiment may be a process for selective hydrogenation of acetylenes and diolefins to olefins. The process may include passing an olefin stream through a demethanizer, thereby creating a demethanized olefin stream, contacting the demethanized olefin stream with a layered catalyst at reaction conditions, and thereby creating an olefin product stream including olefins and paraffins. Generally, the layered catalyst has an inner core including an inert material, an outer layer, including a metal oxide, bonded to the inner core, and a metal, which is an International Union of Pure and Applied Chemistry Group 8-10 metal, deposited on the outer layer. Usually, the layered catalyst has an accessibility index of about 3—about 500, a void space index of about 0—about 1, or both an accessibility index of about 3—about 500 and a void space index about 0—about 1.

Yet a further exemplary embodiment can be a process for selective hydrogenation of acetylenes and diolefins to olefins. The process can include contacting a feedstream having olefins, acetylenes and diolefins with a layered catalyst at reaction conditions thereby creating an output stream with a reduced amount of acetylenes and diolefins. Generally, the layered catalyst has an inner core including an inert material, an outer layer, including a metal oxide, bonded to the inner core, and a metal, which is an International Union of Pure and Applied Chemistry Group 8-10 metal, deposited on the outer layer. Usually, the layered catalyst has a combined index of no more than about 1.5.

The embodiments disclosed herein can provide improved activity and selectivity with respect to selective hydrogenation of acetylene compounds.

### DEFINITIONS

As used herein, the term “stream” can include various hydrocarbon molecules, such as straight-chain, branched, or cyclic alkanes, alkenes, alkadienes, and alkynes, and optionally other substances, such as gases, e.g., hydrogen, or impurities, such as heavy metals, and sulfur and nitrogen compounds. The stream can also include aromatic and non-aromatic hydrocarbons. Moreover, the hydrocarbon molecules may be abbreviated C1, C2, C3 . . . Cn where “n” represents the number of carbon atoms in the one or more hydrocarbon molecules. Furthermore, a superscript “+” or “−” may be used with an abbreviated one or more hydrocarbons notation, e.g., C3⁺ or C3⁻, which is inclusive of the abbreviated one or more hydrocarbons. As an example, the abbreviation “C3⁺” means one or more hydrocarbon molecules of three carbon atoms and/or more.

As used herein, the term “zone” can refer to an area including one or more equipment items and/or one or more sub-zones. Equipment items can include one or more reactors or reactor vessels, heaters, exchangers, pipes, pumps, compressors, and controllers. Additionally, an equipment item, such as a reactor, dryer, or vessel, can further include one or more zones or sub-zones.

As used herein, the term “rich” can mean an amount of at least generally about 50%, and preferably about 70%, by mole, of a compound or class of compounds in a stream.

As used herein, the term “substantially” can mean an amount of at least generally about 80%, preferably about 90%, and optimally about 99%, by mole, of a compound or class of compounds in a stream.

As used herein, the term “accessibility index” may be abbreviated “AI” and is determined by the active surface area of the catalyst divided by the thickness of the catalytically active volume as measured from an exterior to an interior of the catalyst. The AI measures the surface area concentrated in the active zone.

As used herein, the term “void space index” may be abbreviated “VSI” and measures the void volume concentrated in the active zone and indicates volumetric inefficiency of the active zone. Typically, the VSI for a layered catalyst is calculated to include an outer layer. For an unlayered catalyst, the VSI is calculated to include a catalytically active outer surface often impregnated with one or more catalytically and/or promotionally active metals.

As used herein, the term “combined index” may be abbreviated “CI” and can be the sum of a modified AI and VSI.

As used herein, the term “pore volume” can mean the pore volume of the outer layer.

As used herein, the term “effective thickness” can mean a thickness based upon a layer as if a material is uniformly distributed over a surface of an inner core.

As used herein, the terms “olefins” and “alkenes” may be used interchangeably.

As used herein, the terms “ethylene” and “ethene” may be used interchangeably.

As used herein, the terms “acetylene” and “ethyne” may be used interchangeably.

As used herein, the term “International Union of Pure and Applied Chemistry” and its abbreviation “IUPAC” may be used interchangeably.

As used herein, the term “parts per million” may be abbreviated “ppm”. In addition, ppm can be on a weight or volume basis, and be abbreviated as, respectively, “wt.

ppm” and “vol. ppm”.

As used herein, the term “weight percent” may be abbreviated “wt. %”.

As used herein, the term “gas hourly space velocity” may be abbreviated “GHSV”.

As used herein, the term “meter-squared per gram” may be abbreviated “m²/g”.

As used herein, the term “cubic centimeters per gram” may be abbreviated “cc/g”.

### BRIEF DESCRIPTION OF THE DRAWINGS

FIG. 1 is a diagram of the front end use of the catalyst for hydrogenation of acetylene.

FIG. 2 is a diagram of the tail end use of the catalyst for hydrogenation of acetylene.

### DETAILED DESCRIPTION

Generally, ethylene and propylene, light olefin hydrocarbons with two or three carbon atoms per molecule are important chemicals for use in the production of other useful materials, such as, respectively, polyethylene and polypropylene. Polyethylene and polypropylene can be two of the most common plastics found in use today and have a wide variety of uses for both as a material fabrication and as a material for packaging. Other uses for ethylene and propylene may include the production of vinyl chloride, ethylene oxide, ethylbenzene and alcohol. Generally, steam cracking or pyrolysis of hydrocarbons produces most of the ethylene and some propylene. Ethylene can be produced through several means, such as steam cracking of hydrocarbons, catalytic cracking of hydrocarbons, or olefin cracking of larger olefinic feedstocks. However, ethylene for use in the production of polyethylene may be substantially pure. Usually, the methods of producing ethylene generate a product stream with a substantial amount of acetylene, which can be as high as about 2—about 3 volume percent of the ethylene/ethane stream.

Selective hydrogenation of acetylenes and diolefins can improve the quality of the olefin product stream while increasing the amount of olefins is achieved by using a more selective catalyst. The catalyst as described in the embodiments herein may include a material having properties that distinguish it from current commercial catalysts. These properties can be determined from the indexes disclosed herein for choosing a catalyst that has good selectivity in this process. Usually, the catalyst selectively hydrogenates the acetylenes and diolefins to an amount of no more than about 5 wt. ppm of the olefin product stream, and preferably reduces the acetylenes and diolefins to less than about 1 wt. ppm.

Usually, the production of higher olefins also generates diolefins and acetylenes. The purity of olefin streams can affect the quality for products such as detergents and can affect the reactions in alkylation reactions. The catalyst as described in the embodiments herein can also be used for the selective hydrogenation of one or more acetylenes and diolefins having from 2 to 8 carbons atoms to generate higher quality olefin streams.

Generally, the catalyst is a layered catalyst and has an inner core including an inert or solid material. Typically, an outer layer is bonded to the inner core, where the outer layer includes a metal oxide. The catalyst may include a metal selected from an IUPAC Group 8-10 metal, which can be deposited on the outer layer. The catalyst can also have an AI of about 3—about 500, preferably about 3—about 20, and optimally about 4—about 20. The AI may be in units of m²/g, and the formula for calculating AI is provided below.

Desirably, the metal deposited on the outer layer is platinum, palladium, or a mixture thereof, and is deposited in a concentration of about 100—about 50,000 wt. ppm, preferably about 200—about 20,000 wt. ppm of the catalyst.

Generally, the catalyst inner core includes an inert or solid material made up of one or more of the following: a cordierite, a mullite, an olivine, a zirconia, a spinel, a kyanite, an alumina, a silica, an aluminate, a silicate, a titania, a nitride, a carbide, a borosilicate, a boria, an aluminum, a magnesia, a fosterite, a kaolin, a kaolinite, a montmorillonite, a saponite, a bentonite, a clay that has little or low acidic activity, a gamma alumina, a delta alumina, an eta alumina, and a theta alumina. The inner core can have an effective diameter of generally about 0.05—about 10 mm, preferably about 0.8—about 5 mm, and optimally about 0.8—about 3 mm. The effective diameter for non-spherical shapes is the diameter that the shaped particle would have if it were molded into a sphere. In a preferred embodiment, the dried shaped particles are substantially spherical in shape.

The outer layer can be deposited on and bonded to the inner core to an effective thickness of about 1—about 200 micrometers. A preferred outer layer thickness is about 20—about 100 micrometers, with a more preferred outer layer thickness of about 20—about 70 micrometers. The actual thickness can vary somewhat around the particle. The inner core can have an irregular surface and lead to some irregularities in the distribution of the material of the outer layer. The material of the outer layer may be selected from one or more of the following: a gamma alumina, a delta alumina, an eta alumina, a theta alumina, a silica-alumina, a zeolite, a nonzeolitic molecular sieve, a titania, and a zirconia.

In an alternative embodiment, the catalyst may be a layered catalyst having an inner core including an inert material. Generally, an outer layer is bonded to the inner core, where the outer layer includes a metal oxide. Usually, the catalyst includes a metal selected from an IUPAC Group 8-10 metal, which is deposited on the outer layer. The catalyst can also have a VSI of generally no more than about 1, preferably about 0—about 1, more preferably about 0.0001—about 0.9, and optimally about 0.001—about 0.8. The VSI may be in units of (cc* μm)/g, and the formula for calculating VSI is provided below. Additionally, the catalyst may have a CI of generally no more than about 1.5, preferably about 0.5—about 1.5, and optimally about 1.0—about 1.5. The units of CI may be (cc* μm)/g plus m²/g, and the formula for calculating CI is provided below.

The inert inner core may be selected from the materials as mentioned above, and the outer layer can include a material from the list above. The first metal deposited on the outer layer can be selected from the metals listed above.

Control of the selective hydrogenation process can minimize the hydrogenation of olefins, and thus the product loss. This control can be improved by selecting catalysts having an AI greater than about 3 or a VSI of no more than about 1.

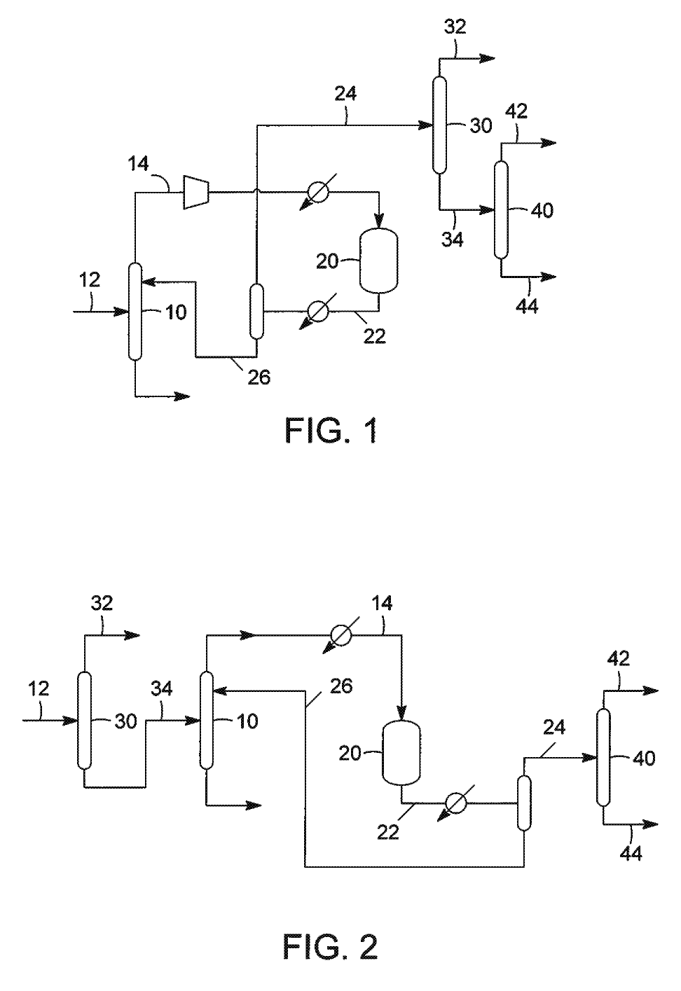

Generally, this catalyst is useful for the selective hydrogenation of acetylenes and diolefins to olefins, and especially acetylene to ethylene, while having minimal side reactions such as hydrogenation of the olefins to paraffins, and especially ethylene to ethane. The process as shown in FIG. 1 can be a front end process. Typically, a process feedstream 12 including ethylene, ethane, and acetylene is passed through a deethanizer 10, and the overhead ethylene rich stream 14 is passed to the selective hydrogenation reactor 20. Usually, the overhead ethylene rich stream 14 is compressed and the temperature adjusted before passing to the selective hydrogenation reactor 20. Generally, temperature adjusting cools the overhead ethylene rich stream 14 that has been compressed. The process for using the catalyst can include contacting the overhead ethylene rich stream 14 having ethylene and acetylene with the catalyst having either an AI of about 3—about 500, a VSI of about 0—about 1, a CI of about 0.5—about 1.5, or a combination of these indexes at reaction conditions. Thus, an ethylene output stream may be created, where the catalyst is as described above. The selective hydrogenation reaction conditions can include a pressure of generally about 0.1—about 14.0 MPa, preferably about 0.5—about 10.0 MPa, and optimally about 0.800—about 7.0 MPa. Usually, the temperature for the selective hydrogenation is about 10—about 300° C., preferably about 30—about 200° C.

The selective hydrogenation conditions may include a hydrogen to acetylene/diolefin molar ratio of about 0.1—about 10,000, preferably about 0.1—about 10, more preferably about 0.5—about 5, and optimally about 0.5—about 3. The source of the process feedstream 12 can be from a catalytic naphtha cracker, and in the process of producing an ethylene rich feedstream, a significant amount of carbon monoxide may be generated. The amount of carbon monoxide can be about 0—about 8,000 vol. ppm, preferably about 0.01—about 10 vol. ppm. When there is a high amount of carbon monoxide, the monoxide may act as a reversible blocker to active catalyst sites. The operating conditions of the selective hydrogenation reactor can include a GHSV of generally about 1,000—about 15,000 hr⁻¹, preferably of about 2,000—about 12,000 hr⁻¹, and optimally about 8,000—about 12,000 hr⁻¹.

Generally, the selective hydrogenation reactor 20 passes an output stream 22 having a reduced acetylene content. The output stream 22 may be cooled and can generate some condensate. Typically, the output stream 22 is separated into a condensate stream 26 which is passed back to the deethanizer 10 as reflux, and into a vapor stream 24. Often, the vapor stream 24 is passed to a demethanizer 30 where the vapor stream 24 is split into a methane rich stream 32 that includes hydrogen and residual carbon monoxide, and an ethane/ethylene stream 34. Usually, the ethane/ethylene stream 34 is passed through a separation process using an ethane/ethylene splitter 40 for separating out the ethane to recover an olefin rich stream including ethylene. An overhead stream 42 including ethylene may be generated at a quality level for use as a polymer feedstock. A bottoms stream 44 including ethane can be directed to other processing units or as an end product.

In another embodiment, the process for the selective hydrogenation of acetylene to ethylene as shown in FIG. 2 may be a tail end process. Typically, a process feedstream 12 is passed through a demethanizer 30, creating an overhead stream 32 including methane and carbon monoxide, and a demethanizer bottoms stream 34 including ethane, ethylene, acetylene and one or more C3⁺ hydrocarbons. Usually, the demethanizer bottoms stream 34 is passed to a deethanizer 10 where the deethanizer splits the demethanizer bottoms stream 34 into a deethanizer overhead, or ethylene, the stream 14 including ethane, ethylene, and acetylene, and a bottoms stream including the one or more C3⁺ hydrocarbons. The deethanizer overhead stream 14 may be passed to a selective hydrogenation reactor 20 where the acetylene is selectively converted to ethylene. The deethanizer overhead or ethylene stream 14 can be temperature adjusted before passing to the selective hydrogenation reactor 20. The selective hydrogenation feed may include an additional hydrogen feedstream as needed. Often, the ethylene stream 14 is contacted with a selective hydrogenation catalyst, having either an AI of about 3—about 500, a VSI of about 0—about 1, a CI of about 0.5—about 1.5, or a combination thereof, within the reactor at reaction conditions, where the catalyst is as described above.

The selective hydrogenation reaction conditions can include a pressure of about 0.1—about 14.0 MPa, preferably about 0.50—about 10 MPa, and optimally about 0.80—about 7.0 MPa. The temperature for the selective hydrogenation may about 10—about 300° C., preferably about 30—about 200° C. The hydrogen to acetylene molar ratio is generally about 0.1—about 20, preferably about 0.1—about 10, more preferably about 0.5—about 5, and optimally about 0.5—about 3. The source of the process feedstream 12 can be from a catalytic naphtha cracker, steam cracker, or olefin cracking unit, and in the process of producing an ethylene rich feedstream, a significant amount of carbon monoxide may be generated. However, with the feedstream passing through the demethanizer 30 before passing to the selective hydrogenation reactor 20, the amount of carbon monoxide can be about 0.1—about 10 vol. ppm. The operating conditions of the selective hydrogenation reactor can include a GHSV of about 1,000—about 5,000 hr⁻¹, preferably no more than about 4,000 hr⁻¹.

Generally, the selective hydrogenation reactor 20 generates a product stream 22 with a reduced acetylene content, and is passed to an ethane/ethylene splitter 40. The product stream 22 may be cooled and can generate some condensate. Typically, the product stream 22 is passed to a vapor-liquid separator where the condensate 26 is recovered and passed back to the deethanizer 10 as reflux. Often, the vapor stream 24 is passed to the ethane/ethylene splitter 40, where the ethane/ethylene splitter 40 generates an overhead stream 42 including ethylene is generated at a quality level for use as a polymer feedstock, and a bottoms stream 44 including ethane is directed to other processing units, or as an end product.

The catalyst for use in the tail end process, that can have the methane and a portion of the carbon monoxide removed before the selective hydrogenation, can be treated with an alkali metal to reduce the acidity of the catalyst. Generally, the catalyst is treated with an alkali metal in an amount less than about 0.5 wt. % of the outer layer, preferably about 0.1—about 0.5 wt. % of the outer layer. Useful alkali metals may include lithium (Li), sodium (Na), potassium (K), rubidium (Rb), and cesium (Cs). While treating with an alkali metal, it is generally molar amounts that give comparable activity, i.e., an atom of Li gives the same response as an atom of K. Therefore, the weight amounts for the lighter lithium may be reduced according to the ratio of the atomic weights. As an example, with Pd-only and Pd/Ag catalysts, about 3,300 wt. ppm K and about 500 wt. ppm Li have similar activities and selectivities.

However, the addition of an alkali metal may indicate increased activity, but decreased selectivity for front end catalysts. For catalysts tested having Pd only on the outer layer, lower potassium can provide higher activity and selectivity, or lower ethane formation. This can demonstrate preferential acetylene hydrogenation over ethylene hydrogenation, and lithium gives higher activity, but lower selectivity. For catalysts tested having Pd/Ag on the outer layer, lower potassium may also provide higher activity and lower selectivity.

Several indexes, as described above, can be used to identify catalyst having improved activity and selectivity for hydrogenating acetylene compounds. These indexes are the AI, VSI, and CI.

The formula for calculating the AI in Examples 1-17 is as follows:

AI=((SATH))*((IZT*1000+2*AZT)/100)

where:
IZT=the inert zone thickness of the catalyst in mm;
AZT=the active zone thickness of the catalyst in μm; and
SATH=the active surface of the entire catalyst divided by its thickness and calculated by the formula:

SATH=(Total SA)*((% SA in AZ)/AZT/100)

where:

Total SA=(MSA)*(AZ/100)+(ISA)*(1−AZ/100)

where:

MSA=the surface area of the active material in m²/g;

AZ=the active zone of the catalyst in percent; and

ISA=the surface area of the inert zone material in m²/g;

The AZ is calculated as:

AZ=(1−((IZT/2)³/(IZT/2+(AZT/1000))³))*100

where: IZT and AZT are defined above.

where:

% SA in AZ=(MSA)*((AZ)/100/(Total SA))*100

where: MSA, AZ, and Total SA are defined above.

The VSI calculations for Examples 1-12 utilize a reference value of the active zone to calculate the active portion of the catalyst. The VSI is calculated for layered catalysts using the following formula:

VSI=10*(PV)*(APR/AZT)*(AZ)/(REF)*((IZT*1000+2*AZT)/1000)

where:
PV=the pore volume of the outer layer of the REF catalyst in cc/g;
APR=the average pore radius of the catalyst in nm;
AZT is as defined above;
IZT is as defined above;
AZ is as defined above; and
REF=the active zone of a reference catalyst, namely Examples 3 and 9, having an active zone thickness of 25 μm and is a fixed AZ value of 4.8378% for gamma alumina in Examples 1-6 and theta alumina in Examples 7-12. Thus, the reference value is 4.8378% for Examples 1-12 and the Examples 1-2, 4-8, and 10-12 are scaled to the REF value for determining their VSI. The REF values are the calculated AZ values for Examples 3 and 9.

The APR is calculated as follows:

APR=2000*(PV/SA)

where SA is the surface area based on the whole catalyst in m²/g. In Examples 1-12, the PV is the pore volume in cc/g and the SA is the surface area in m²/g of the reference catalyst. Specifically, the PV is 0.033 cc/g and the SA is 11 m²/g for Examples 1-6 and the PV is 0.040 cc/g and the SA is 5 m²/g for Examples 7-12.

The reference active zones are not utilized for calculating the VSI for unlayered comparative catalysts of Examples 13-17. Rather, a separate formula is utilized for calculating the VSI for the unlayered, comparative Examples 13-17:

VSI=10*PV*AZ*(APR/AZT/100)*((IZT*1000+2*AZT)/1000)

where:
APR, AZ, IZT, and AZT are defined as above. The APR is calculated as above, but for the unlayered catalyst the MSA and SA have the same values. Moreover, PV is the pore volume of the catalyst (0.29 g/cc) as the active zone and inactive zone have the same material values.

The CI is defined by the formula:

CI=VSI+AI/10

The AI can be divided by 10 to provide a modified AI to correlate VSI and AI.

### Illustrative Embodiments

The following examples are intended to further illustrate the subject catalyst. These illustrations of embodiments of the invention are not meant to limit the claims of this invention to the particular details of these examples. These examples are based on engineering calculations and actual operating experience with similar processes.

Several catalysts are tested and can have the following properties as depicted in TABLES 1 and 2:

TABLE 1

| Catalyst | | | | | |
|---|---|---|---|---|---|
| Sample | AZT | Active Zone | MSA | IZT | ISA |
| Number | μm | Material | m²/g | mm | m²/g |
| 1 | 5.0 | gamma alumina | 208 | 3 | 3 |
| 2 | 12.5 | gamma alumina | 208 | 3 | 3 |
| 3 | 25.0 | gamma alumina | 208 | 3 | 3 |
| 4 | 50.0 | gamma alumina | 208 | 3 | 3 |
| 5 | 100.0 | gamma alumina | 208 | 3 | 3 |
| 6 | 200.0 | gamma alumina | 208 | 3 | 3 |
| 7 | 5.0 | theta alumina | 96 | 3 | 3 |
| 8 | 12.5 | theta alumina | 96 | 3 | 3 |
| 9 | 25.0 | theta alumina | 96 | 3 | 3 |
| 10 | 50.0 | theta alumina | 96 | 3 | 3 |
| 11 | 100.0 | theta alumina | 96 | 3 | 3 |
| 12 | 200.0 | theta alumina | 96 | 3 | 3 |
| 13 | 25.0 | alpha alumina | 5 | 3 | 5 |
| 14 | 50.0 | alpha alumina | 5 | 3 | 5 |
| 15 | 100.0 | alpha alumina | 5 | 3 | 5 |
| 16 | 200.0 | alpha alumina | 5 | 3 | 5 |
| 17 | 300.0 | alpha alumina | 5 | 3 | 5 |

TABLE 2

| Catalyst | Inert | | | | |
|---|---|---|---|---|---|
| Sample | Zone | SA | | APR | PV |
| Number | Material | m²/g | AZ % | nm | cc/g |
| 1 | cordierite | 11 | 0.9934 | 6 | 0.033 |
| 2 | cordierite | 11 | 2.4589 | 6 | 0.033 |
| 3 | cordierite | 11 | 4.8378 | 6 | 0.033 |
| 4 | cordierite | 11 | 9.3686 | 6 | 0.033 |
| 5 | cordierite | 11 | 17.6025 | 6 | 0.033 |
| 6 | cordierite | 11 | 31.3047 | 6 | 0.033 |
| 7 | cordierite | 5 | 0.9934 | 16 | 0.040 |
| 8 | cordierite | 5 | 2.4589 | 16 | 0.040 |
| 9 | cordierite | 5 | 4.8378 | 16 | 0.040 |
| 10 | cordierite | 5 | 9.3686 | 16 | 0.040 |
| 11 | cordierite | 5 | 17.6025 | 16 | 0.040 |
| 12 | cordierite | 5 | 31.3047 | 16 | 0.040 |
| 13 | alpha alumina | 5 | 4.8378 | 116 | 0.290 |
| 14 | alpha alumina | 5 | 9.3686 | 116 | 0.290 |
| 15 | alpha alumina | 5 | 17.6025 | 116 | 0.290 |
| 16 | alpha alumina | 5 | 31.3047 | 116 | 0.290 |
| 17 | alpha alumina | 5 | 42.1296 | 116 | 0.290 |

In Examples 1-6, Example 3 is used as a standard, and the total surface area (SA) and inert surface area (ISA) are determined by nitrogen gas adsorption according to ASTM D3663-03 (2008), and the pore volume (PV) for the outer layer of gamma alumina is determined by ASTM-D4222-03 (2008) for a 25 μm AZT catalyst. The SA is determined to be 11 m²/g, and is divided by the weight fraction of 5.3 wt. % alumina (0.053) to calculate a value of 208 m²/g for the MSA. This value of 208 m²/g is utilized for Examples 1-6. The MSA and PV for the AZ are multiplied by the volume fraction of the AZ relative to the entire catalyst pellet or sphere. These values are used to calculate a reference value and are scaled to determine the other thicknesses scaled proportionally to the layer volume for calculating VSI for Examples 1-2 and 4-6.

In Examples 7-12, Example 9 is used as a standard and the SA and ISA are determined by nitrogen gas adsorption according to ASTM D3663-03 (2008), and the pore volume (PV) for the outer layer of gamma alumina is determined by ASTM-D4222-03 (2008) for a 25 μm AZT catalyst. The SA is determined to be 5 m²/g, and is divided by the weight fraction of 5.2 wt. % alumina (0.052) to arrive at a value of 96 m²/g for the MSA. This value of 96 m²/g is utilized for Examples 7-12. The MSA and PV for the AZ are multiplied by the volume fraction of the AZ relative to the entire catalyst pellet or sphere. These values are used to calculate a reference value and are scaled to determine the other thicknesses scaled proportionally to the layer volume for calculating VSI for Examples 7-8 and 10-12.

Alternatively, the MSA and ISA can be calculated by the method of Brunauer, Emmett, and Teller by measuring the quantity of argon adsorbed on the catalyst at −183° C. with the cross-sectional area of the argon atom being taken as 14.4 square Angstrom units. The PV may be calculated by determining the “mercury density” and the “helium density”. The mercury density is determined by immersing a support in mercury at 20° C. and 120 kPa pressure, under which conditions about 15 minutes are allowed for attainment of equilibrium. The helium density is determined by immersing the support in helium at room temperature. The pore volume per gram can be determined by subtracting the reciprocal of the helium density from the reciprocal of the mercury density. These alternative methodologies are disclosed in, e.g., U.S. Pat. No. 4,404,124.

In comparative Examples 13-17, the MSA and PV are obtained by taking the median value of typical aluminas disclosed in U.S. Pat. No. 4,404,124. Particularly, column 2, lines 26-42 discloses typical aluminas having ranges of a surface area of about 3—about 7 m²/g and a pore volume of about 0.24—about 0.34 cc/g. Thus, median values of 5 m²/g and 0.29 cc/g for, respectively, MSA and PV are utilized in Examples 13-17.

To determine the AZT and IZT in Examples 1-17, the catalyst is mounted, ground, and polished to a maximum diameter and the change of composition between the outer layer and inner core is identified by using a scanning electron microscope with energy-dispersive X-ray spectroscopy (may be abbreviated SEM/EDS). The change in composition from alumina in the outer layer to silica and magnesia in the core is identified for determining the boundary between the outer layer and inner core. Alternatively, an effective depth of a deposited metal drops below a predetermined cutoff, e.g., 10%, by weight, for determining the thickness of the outer layer. If the outer layer is very thin, a relationship between the weight of the layer added and the thickness for the thicker layers may be used to obtain an effective layer density. In yet another procedure, an agent can be utilized to stain the deposited metal to highlight the boundary between the outer layer and inner core, as disclosed in U.S. Pat. No. 4,404,124.

TABLE 3 compares the layered exemplary catalysts having layer thicknesses from 5 to 200 micrometers of either gamma or theta alumina with a conventional comparative catalyst prepared on an alpha alumina. The comparative catalyst has its surface impregnated to various depths from 25-300 μm. All catalysts are assumed to be 3 mm spheres for a common basis of presentation. The parameters indicate why very thin active zones are not practical for conventional catalysts. The active zones are defined as the region in which at least 90% of the active metal/active sites occur. Typical loadings provide a very high percent of monolayer coverage that may yield poor metal utilization and often have very large metal particle agglomerates.

TABLE 3

| Catalyst | | | CI |
|---|---|---|---|
| Sample | AI | VSI | (cc* μm)/g |
| Number | m²/g | (cc* μm)/g | plus m²/g |
| 1 | 12.44 | 0.245 | 1.49 |
| 2 | 12.38 | 0.244 | 1.48 |
| 3 | 12.28 | 0.242 | 1.47 |
| 4 | 12.08 | 0.238 | 1.45 |
| 5 | 11.72 | 0.231 | 1.40 |
| 6 | 11.07 | 0.218 | 1.33 |
| 7 | 5.74 | 0.791 | 1.37 |
| 8 | 5.71 | 0.787 | 1.36 |
| 9 | 5.67 | 0.781 | 1.35 |
| 10 | 5.58 | 0.768 | 1.33 |
| 11 | 5.41 | 0.745 | 1.29 |
| 12 | 5.11 | 0.704 | 1.22 |
| 13 | 0.30 | 1.99 | 2.02 |
| 14 | 0.29 | 1.95 | 1.98 |
| 15 | 0.28 | 1.89 | 1.92 |
| 16 | 0.27 | 1.79 | 1.82 |
| 17 | 0.25 | 1.70 | 1.73 |

The exemplary embodiments use gamma and theta alumina for the outer layer of the catalyst and have various effective thicknesses. Typically, the exemplary catalysts have a high accessibility index, greater than about 3, and a low void space index, less than about 1, relative to a standard commercial catalyst using alpha alumina. Often, conventional catalysts using alpha alumina have very large average pore diameters.

Although not wanting to be bound by theory, the indexes can indicate why thin active zones are not practical for conventional catalysts. The active zones can be regions in which greater than 90% of the active metal sites occur. Typically, conventional catalysts with thin active zones yield poor metal utilization due to the fact that they have very high percent monolayer coverage and large metal particle agglomerates. Changing the pore size of the catalyst can improve the performance of the selective hydrogenation for the front end process.

From the tests, catalyst activity tends to increase for catalysts with outer layer effective thicknesses in the range of 5 to 50 micrometers. This suggests thinner layers will give better performance. The exemplary catalysts allow for thinner layers with lower metal deposition. This has the potential to reduce the tendency to accumulate heavy by-products and thereby reduce the deactivation of the catalyst.

### Catalyst Preparation Procedure:

The catalyst is prepared by adding a solution of the appropriate metal salts, typically nitrates, to the desired amount of support. In particular, a 1% HNO₃ solution, relative to the support weight, is diluted with deionized water to provide a volume of solution approximately equivalent to the support volume, or a 1:1 solution to support volume ratio. The solution is contacted with the support at room temperature for one hour with constant agitation, or rolling to ensure good support and solution contact. The solution is then heated to 100° C. and the liquid is evaporated over a period of time that is greater than 3 hours, thereby creating the impregnated support. The final support should be “free-rolling” or freely moving in the container. The final moisture content varies with the specific support, but is typically in the range of 20 to 30 wt %.

The impregnated support is then transferred to a container suitable for calcination and reduction. The support is dried at 120° C. in flowing dry air for 3 hours, then ramped up to 450° C. in flowing dry air at a rate of 5° C/min and held at 450° C. for 1 hour. Alternatively, the calcination temperature may be ramped up to 600° C. with steam of up to 3%, by volume, water. The sample is cooled to room temperature.

For reduction, the sample is ramped up to 200° C. in flowing dry N₂ at a rate of 5°

C/min, and held at 200° C. for one hour. Alternatively, the reduction temperature may be ramped up to 350° C. The flowing dry N₂ is shut off and hydrogen is then flowed over the catalyst and held for 3 hours. The hydrogen is then switched to nitrogen and the catalyst sample is cooled to room temperature.

For a two step procedure, the calcined and reduced catalyst from the first step is used as the support for the second step, and the typical impregnation, drying, calcination and reduction steps are followed with the second set of metal salts in solution.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing, all temperatures are set forth in degrees Celsius and, all parts and percentages are by weight, unless otherwise indicated.

While the invention has been described with what are presently considered the preferred embodiments, it is to be understood that the invention is not limited to the disclosed embodiments, but it is intended to cover various modifications and equivalent arrangements included within the scope of the appended claims.

1. A process for selective hydrogenation of acetylenes and diolefins to olefins, comprising:
A) contacting a feedstream comprising olefins, acetylenes and diolefins with a layered catalyst at reaction conditions thereby creating an output stream with a reduced amount of acetylenes and diolefins, wherein the layered catalyst comprises:
1) an inner core comprising an inert material;
2) an outer layer bonded to the inner core, wherein the outer layer comprises a metal oxide; and
3) a metal deposited on the outer layer, wherein the metal is an International Union of Pure and Applied Chemistry Group 8-10 metal;
4) wherein the layered catalyst has an accessibility index of about 3—about 500, a void space index about 0—about 1, or both an accessibility index of about 3—about 500 and a void space index of about 0—about 1.

2. The process of claim 1, wherein selective hydrogenation conditions comprise a pressure of about 0.1—about 14.0 MPa.

3. The process of claim 2, wherein the selective hydrogenation conditions comprise a pressure of about 0.5—about 10.0 MPa.

4. The process of claim 3, wherein the selective hydrogenation conditions comprise a pressure of about 0.8—about 7.0 MPa.

5. The process of claim 1, wherein selective hydrogenation conditions comprise a temperature of about 10—about 300° C.

6. The process of claim 5, wherein the selective hydrogenation conditions comprise a temperature of about 30—about 200° C.

7. The process of claim 1, wherein selective hydrogenation conditions include a hydrogen to acetylene/diolefin molar ratio of about 0.1—about 10,000.

8. The process of claim 1, wherein the feedstream comprises about 0—about 8,000 vol. ppm of carbon monoxide.

9. The process of claim 1, wherein the layered catalyst is used for selective hydrogenation of one or more acetylenes and diolefins having from 2 to 8 carbons.

10. The process of claim 1, further comprising a separation process for recovering an olefin rich stream.

11. A process for selective hydrogenation of acetylenes and diolefins to olefins, comprising:
A) passing an olefin stream through a demethanizer, thereby creating a demethanized olefin stream;
B) contacting the demethanized olefin stream with a layered catalyst at reaction conditions, wherein the layered catalyst comprises:
1) an inner core comprising an inert material;
2) an outer layer bonded to the inner core, wherein the outer layer comprises a metal oxide; and
3) a metal deposited on the outer layer, wherein the metal is an International Union of Pure and Applied Chemistry Group 8-10 metal;
4) wherein the layered catalyst has an accessibility index of about 3—about 500, a void space index of about 0—about 1, or both an accessibility index of about 3—about 500 and a void space index about 0—about 1;
C) thereby creating an olefin product stream comprising olefins and paraffins.

12. The process of claim 11, wherein the layered catalyst has been treated with an alkali metal to reduce an acidity of the catalyst.

13. The process of claim 11, wherein selective hydrogenation conditions comprise a pressure of about 0.1—about 14.0 MPa.

14. The process of claim 13, wherein the selective hydrogenation conditions comprise a pressure of about 0.8—about 7.0 MPa.

15. The process of claim 11, wherein selective hydrogenation conditions comprise a temperature of about 10—about 300° C.

16. The process of claim 15, wherein the selective hydrogenation conditions comprise a temperature of about 30—about 200° C.

17. The process of claim 11, wherein selective hydrogenation conditions include a hydrogen to acetylene/diolefin molar ratio of about 0.1—about 10,000.

18. The process of claim 11, wherein the feedstream comprises about 0—about 8,000 vol. ppm of carbon monoxide.

19. The process of claim 11, wherein the feedstream comprises about 0.01—about 10 ppm vol. ppm of carbon monoxide.

20. A process for selective hydrogenation of acetylenes and diolefins to olefins, comprising:
A) contacting a feedstream comprising olefins, acetylenes and diolefins with a layered catalyst at reaction conditions thereby creating an output stream with a reduced amount of acetylenes and diolefins, wherein the layered catalyst comprises:
1) an inner core comprising an inert material;
2) an outer layer bonded to the inner core, wherein the outer layer comprises a metal oxide; and
3) a metal deposited on the outer layer, wherein the metal is an International Union of Pure and Applied Chemistry Group 8-10 metal;
4) wherein the layered catalyst has a combined index of no more than about 1.5.
